(19) United States
(12) Patent Application Publication
(10) Pub. No.: US 2025/0339588 A1
(43) Pub. Date: Nov. 6, 2025

(54) MANUFACTURE AND USE OF MEDICAL DEVICE COATINGS

(71) Applicant: MicroVention, Inc., Aliso Viejo, CA (US)

(72) Inventors: Nicholas Davis, Mission Viejo, CA (US); Bryan Kan, Orange, CA (US); Joseph Gulachenski, Trabuco Canyon, CA (US)

(21) Appl. No.: 18/866,363

(22) PCT Filed: May 16, 2023
(86) PCT No.: PCT/US2023/067044

§ 371 Date: Nov. 15, 2024

Related U.S. Application Data
(60) Provisional application No. 63/342,321, filed on May 16, 2022.

(51) Int. Cl. A61L 29/08; C09D 133/08

(52) U.S. Cl. CPC A61L 29/085; C09D 133/08; A61L 2400/10; A61L 2420/08

(57) ABSTRACT

Lubricious coatings for medical devices and applying them to a specific section of a catheter are described herein.

### CROSS REFERENCE TO RELATED APPLICATION

This application claims the benefit of, and priority to, U.S. Provisional Patent Application Ser. No. 63/342,321, filed on May 16, 2022, the entire disclosure of which is hereby incorporated by reference in its entirety.

### FIELD

Described herein are coatings for medical devices and methods of applying those coatings.

### BACKGROUND

Tubular medical devices such as catheters and microcatheters are used to conduct diagnostic and therapeutic endovascular interventions. Catheters are often formed of thermoplastic polymers that have high frictional forces. These high frictional forces make vascular navigation difficult. For example, when using a support catheter, friction is felt when advancing the guide catheter (or balloon catheter) over the support catheter. The maximum amount of friction is felt around pre-shaped (such as angled or “bent”) sections of the inner support catheter.

Hydrophilic coatings along the entire distal section of the catheter can reduce friction, but can cause issues of reduced stability.

### SUMMARY

Disclosed herein are medical device coatings and methods of their manufacture and use. Disclosed coatings include “lubricious” or friction-reducing coatings for medical devices, thereby increasing ease of use and enabling more precise therapeutic intervention.

Applying lubricious coating to sections (such as constricted, bent, or angled sections) of medical devices significantly reduces the friction created when, for example, advancing the guide or balloon catheter over the inner support catheter. Providing a catheter or microcatheter with a lubricious coating at a specific section as described herein would be useful and beneficial.

Thus, by applying the lubricous coating on a specific section/particular bulbous section of the catheter and not a distal section of the catheter, friction is reduced while maintaining stability. For example, applying the lubricous coating to a particular section of a catheter results in good stability and low friction when advancing the guide or balloon catheter over the catheter.

The herein described coatings can be applied to medical devices such as medical devices that can be subjected to human tissues. In some embodiments, the coatings can be applied to medical devices that are used inside the body, for example, vessels or other lumens. In some embodiments, the vessels can be blood vessels. In some embodiments, the medical devices can be catheters or microcatheters.

In embodiments, the coatings can be synthetic and durable and lubricious. In some embodiments, the coatings can be ultra-violet (UV) cured. In embodiments, the coatings can be applied to the interior, the exterior, or both of a device.

Lubricious coatings can reduce and/or minimize frictional forces between a medical device, such as a catheter or microcatheter, and a vessel wall, thereby enhancing trackability of the medical device throughout the vasculature. In embodiments, catheter surfaces are modified with lubricious coatings to reduce the frictional forces and enhance the ability of the catheter to be advanced through tortuous and distal vasculature.

In some embodiments, the herein described coatings can comprise a single layer. In some embodiments, the herein described coatings can comprise two layers, for example a base coat and a top coat. The base coat functions as a tie layer between the catheter's thermoplastic polymer surface and the top coat. The base coat is designed to adhere to the catheter and provide binding sites for the attachment of the top coat. The top coat is designed to adhere to the base coat and provide lubricity to reduce the frictional forces created when the catheter is moved in the vasculature.

In embodiments, disclosed coatings can generally comprise a base coat including a copolymer of a first tetrahydrofuryl acrylate monomer and a second monomer including a functional group amenable to further derivatization and plurality of reactive moieties, and a top coat including a hydrophilic polymer containing more than two reactive moieties per molecule.

Methods of coating a thermoplastic surface, such as a catheter or microcatheter surface, are also described. The methods can comprise, for example, applying a base coat comprising a copolymer of a first tetrahydrofuryl acrylate monomer and a second monomer to the thermoplastic surface, and applying a top coat to the base coat, wherein the top coat includes a hydrophilic polymer.

In some embodiments, the coatings described herein are applied to a specific section or sections of the device, for example a catheter. In other embodiments, the coatings described herein are applied to a particular angled, constricted, or bulbous section of the catheter. Application of the coatings to a specific section or particular bulbous section of the catheter reduces friction while maintaining stability. In some embodiments, the coating is not applied to a distal end of the catheter.

### BRIEF DESCRIPTION OF THE DRAWINGS

Aspects, features, and advantages of which embodiments of the invention are capable of will be apparent and elucidated from the following description of embodiments, reference being made to the accompanying drawings, in which:

FIG. 1 illustrates the use of an inner support catheter to guide a guide or balloon catheter to a target location. FIG. 1A illustrates a shaped section of the inner support catheter reformed inside an aortic branch. FIG. 1B illustrates the inner support catheter being pulled back and advanced into a desired vessel. FIG. 1C illustrates a guide or balloon catheter being advanced over the inner support catheter. FIG. 1D illustrates the guide or balloon catheter being further advanced to a target location.

FIG. 2 illustrates the use of an inner support catheter to guide a guide or balloon catheter to a target location. FIG. 2A illustrates the inner support catheter being advanced further into the desired location to provide more support. FIG. 2B illustrates the guide or balloon catheter being advanced to a target location.

FIG. 3 illustrates an inner support catheter used to guide a procedural catheter.

FIG. 4 illustrates the “wavy” shaped section of the inner support catheter of FIG. 3 when it is straightened out.

FIG. 5 illustrates the friction created against the inner liner of the guide or balloon catheter.

FIG. 6 illustrates the coating of the inner support catheter at the specific section of the catheter where friction was observed in FIG. 5.

FIG. 7 illustrates a perspective view of the coated section in FIG. 6.

FIG. 8 illustrates an inner support catheter in an aortic arch. FIG. 8A illustrates an inner support catheter coated at its distal end. FIG. 8B illustrates the inner support catheter having reduced stability.

### DETAILED DESCRIPTION

Described herein are coatings for medical devices and methods of preparation, application, and use. In some embodiments, the coatings can increase the lubricity of the medical device.

### Devices

Disclosed medical devices suitable for use with disclosed coatings can comprise catheters and microcatheters, for example catheters and microcatheters that are formed at least partially of thermoplastic polymers/materials. The thermoplastic polymers can comprise, for example, poly(amides), poly(ethylene terephthalate), poly(urethanes), poly(ether sulfones), poly(carbonates), poly(vinyl chloride), copolymers thereof, and derivatives thereof.

These thermoplastic polymers can have high frictional forces. These high frictional forces make vascular navigation difficult. Thus, the herein described coatings can increase lubricity of the thermoplastic polymer surfaces.

In some embodiments, the coatings described herein can be applied to catheters including, but not limited to, access catheters, support catheters, inner catheters, inner support catheters, and/or a combination thereof. In embodiments, the coating can be applied to a specific section or portion of the catheter. In other embodiments, the coatings described herein can be applied to a constricted, angled, or bulbous section of a catheter. In some embodiments, the coatings described herein can be applied to a non-bulbous section of a catheter. In other embodiments, coatings described herein are applied to an inner support catheter. In some embodiments, the inner support catheter can be used in conjunction with a procedural catheter. Procedural catheters include, but are not limited to, a guide catheter, and/or a balloon catheter. In some embodiments, the inner support catheter can be used in conjunction with one or more procedural catheter(s). The inner support catheter can be used to guide a balloon catheter and/or guide catheter to a target location.

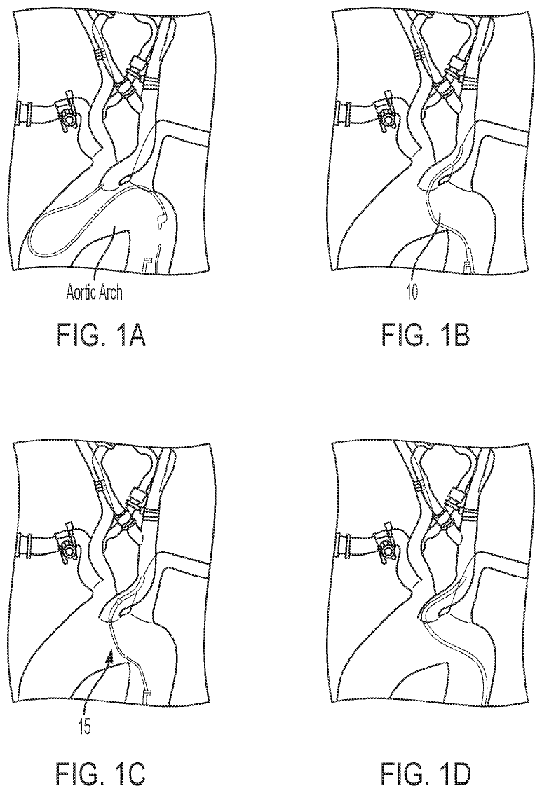

Turning to the Figures, FIG. 1A illustrates a shaped section of the inner support catheter reformed inside an aortic branch. FIG. 1B illustrates the inner support catheter 10 being pulled back and advanced into a desired vessel. FIG. 1C illustrates a guide or balloon catheter 15 being advanced over the inner support catheter. FIG. 1D illustrates the guide or balloon catheter being further advanced to a target location. In some embodiments, a shaped section of the inner support catheter can be reformed inside an aortic branch as depicted in FIG. 1A. The inner support catheter can be pulled back and advanced into a desired vessel as illustrated in FIG. 1B. FIG. 1C illustrates a guide or balloon catheter being advanced over the inner support catheter. The guide or balloon catheter is then further advanced to a target location as depicted in FIG. 1D.

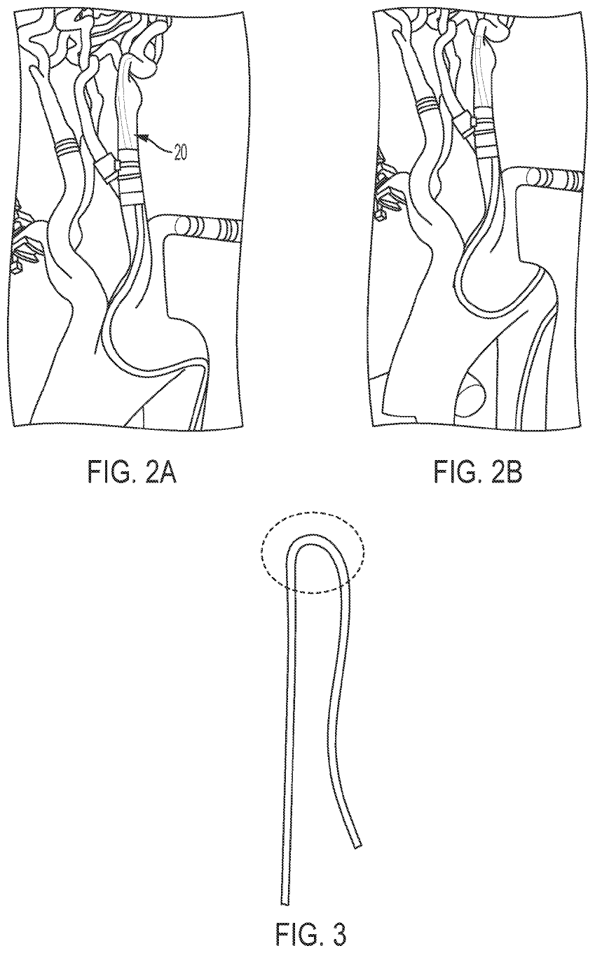

FIG. 2A illustrates the inner support catheter 20 being advanced further into the desired location to provide more support. FIG. 2B illustrates the guide or balloon catheter being advanced to a target location. A shaped section of the inner support catheter can be reformed inside an aortic branch as illustrated. The inner support catheter can be pulled back and advanced into a desired vessel. The inner support catheter can then be advanced further into the desired location to provide more support as illustrated in FIG. 2A. The guide or balloon catheter is then advanced to a target location as depicted in FIG. 2B.

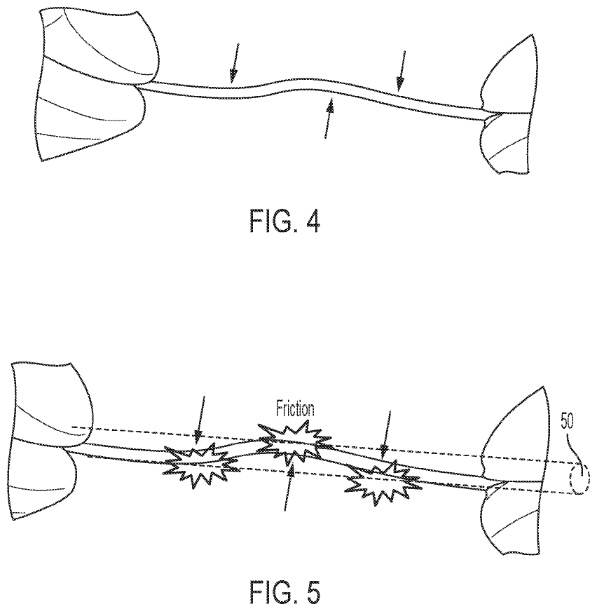

FIG. 3 illustrates an inner support catheter used to guide a procedural catheter. The circled area indicates a region with potentially increased friction. When the inner support catheter is straightened out, e.g. as illustrated in FIG. 4, the shaped section can be wavy.

FIG. 4 illustrates the “wavy” shaped section of the inner support catheter of FIG. 3 when it is straightened out.

FIG. 5 illustrates the friction created against the inner liner of the guide or balloon catheter 50. To reduce friction, the coatings described herein are applied to the inner support catheter. In some embodiments, the coatings are applied to the specific section on the inner support catheter where the friction is observed.

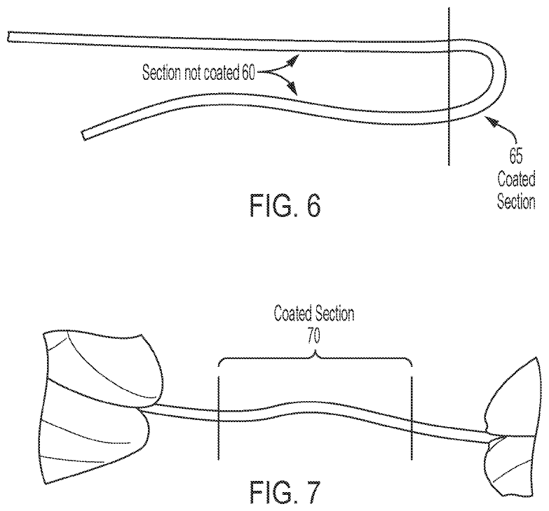

FIG. 6 illustrates the coating of the inner support catheter 65 at the specific section of the catheter where friction was observed in FIG. 5, while uncoated regions are shown at 60.

FIG. 7 illustrates a perspective view of the coated section 70 in FIG. 6.

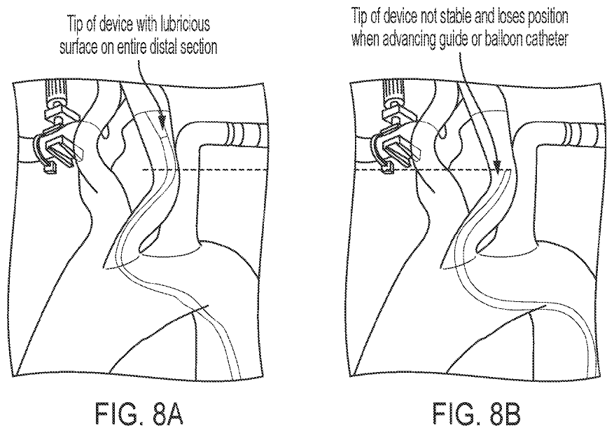

FIG. 8 illustrates an inner support catheter in an aortic arch. FIG. 8A illustrates an inner support catheter coated at its distal end. As demonstrated in FIG. 8A the catheter is above the dotted line. FIG. 8B illustrates the inner support catheter having reduced stability when advancing a guide or balloon catheter. The tip of the catheter is not stable in FIG. 8B as it has fallen below the dotted line in comparison to FIG. 8A.

### Medical Device Coatings

In some embodiments, disclosed coatings can comprise, for example, multiple coats, such as, for example, a base coat and a top coat. In embodiments the base coat functions as a “tie” layer between the catheter's thermoplastic polymer and the top coat. In embodiments, the base coat is designed to adhere to the catheter and provide binding sites for the attachment of the top coat. In embodiments, the top coat is designed to adhere to the base coat and provide lubricity to reduce the frictional forces created when the catheter is moved in the vasculature.

In some embodiments, the base coat comprises a polymer that is a copolymer of a first tetrahydrofurfuryl acrylate monomer and at least one other monomer with functional groups capable of further chemical reaction such as hydroxyl, amine, and carboxylic acid groups. In some embodiments, the at least one other monomer including hydroxyl groups can be hydroxyethyl methacrylate, hydroxyethyl acrylate, hydroxypropyl acrylate, hydroxypropyl methacrylate, hydroxybutyl acrylate, hydroxybutyl methacrylate, combinations thereof, and derivatives thereof. In some embodiments, the at least one other monomer including amine groups can be N-(3-aminopropyl) methacrylamide, 2-aminoethyl methacrylate, 2-aminoethyl methacrylamide, combinations thereof, and derivatives thereof. In some embodiments, the at least one other monomer including carboxylic acids can be acrylic acid, methacrylic acid, beta-carboxyethyl acrylate, combinations thereof, and derivatives thereof.

### Methods of Coating Preparation

In embodiments, to prepare the base coat copolymer the two or more monomers and optionally an initiator can be dissolved in a solvent. The solvent can comprise any solvent that dissolves the two or more monomers and the optional initiator. Solvents can include benzene, toluene, xylene, dimethylformamide, dimethyl sulfoxide, dioxane, 2-methyltetrahydrofuran, anisole, benzonitrile, chlorinated aromatic solvents, diisopropyl ether, diglyme, butanol, and combinations thereof.

Initiators can be used to start the polymerization of the monomers in the solution. In embodiments the polymerization can be initiated by reduction-oxidation, radiation, heat, or any other method known in the art. Radiation cross-linking of the monomers in solution can be achieved with ultraviolet light or visible light with suitable initiators or ionizing radiation (e.g. electron beam or gamma ray) without initiators. Polymerization can be achieved by application of heat, either by conventionally heating the solution using a heat source such as a heating well, or by application of infrared light to the monomers in solution.

In some embodiments, the initiator is azobisisobutyronitrile (AIBN) or a water soluble AIBN derivatives (2,2′-azobis(2-methylpropionamidine) dihydrochloride), or 4,4′-azobis(4-cyanopentanoic acid). Other initiators can comprise N,N,N′,N′-tetramethylethylenediamine, ammonium persulfate, benzoyl peroxides, and combinations thereof, including azobisisobutyronitriles.

In some embodiments, the initiator concentration can be from, for example, about 0.25% w/w to about 2% w/w of the mass of the monomers in solution.

In some embodiments, the polymerization reaction can be performed at elevated temperatures, such as in the range from, for example, about 65° C. to about 85° C.

After the polymerization is completed, the copolymer can be recovered by precipitation in a non-solvent and dried under vacuum.

In embodiments the resulting copolymer can have a molecular weight between about 15,000 g/mole and about 150,000 g/mole or between 25,000 g/mole to 100,000 g/mole. This molecular weight can be derived by gel permeation chromatography with polystyrene standards.

Following polymerization, reactive groups, such as acrylates and/or methacrylates, are added to the copolymer via the hydroxyl, amine, and/or carboxylic acid groups of the second or more monomers. In general, the derivatization compound is a hetero-bifunctional compound. One moiety reacts with the hydroxyl, amine, and/or carboxylic acid groups of the copolymer. The other moiety is an acrylate or methacrylate group. Suitable derivatization compounds include 2-isocyanatoethyl acrylate, 2-isocyanatoethyl methacrylate, acrylic acid N-hydroxysuccinimide ester, methacrylic acid N-hydroxysuccinimide ester, hetero-bifunctional poly(ethylene glycol) with acrylate and isocyanate groups, combinations thereof, and derivatives thereof.

In embodiments, to prepare the derivatized copolymer, the copolymer, and derivatization compound, and optionally any catalyst, can be dissolved in a solvent. In general, any solvent that dissolves the components can be used. Solvents can comprise dimethyl formamide, dimethyl sulfoxide, toluene, acetone, acetonitrile, N,N-dimethylacetamide, N-methyl-2-pyrrolidone, and combinations thereof.

In embodiments, when reacting a derivatization with a nucleophilic group of the base coat copolymer, the molar equivalent of derivatization agent can range from about 5% to about 80% or about 10% to about 50% of the available nucleophilic groups. This level of derivatization corresponds to a range of 4 to 50 reactive groups per molecule. Further, in some embodiments, a Lewis base can be added of as a catalyst. Lewis bases can include triethylamine and pyridine. The Lewis base can be provided at a concentration of, for example, about 1% to about 10% of the moles of the derivatization compound added.

In embodiments the reaction can proceed at elevated temperature, such as about, for example, 30° C., 35° C., 40° C., 45° C., 50° C., or more to form the base coat. After the derivatization is complete, the completed, decorated copolymer can be recovered by precipitation in a non-solvent and dried under vacuum.

In embodiments the top coat can be formed atop the base coat. The top coat polymer can comprise a core, hydrophilic polymer that is derivatized with polymerizable groups. The core hydrophilic polymer can be any naturally-occurring or synthetic polymer, derivatives thereof and combinations thereof. In some embodiments, the core hydrophilic polymer is at least to some degree, soluble in water.

The structure of the core hydrophilic polymer can be linear or branched, including graft, star, comb, brush, and dendrimer structures.

Polymers used for the top coat can comprise, but are not limited to naturally-occurring polymers such as proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, and dextran.

Polymers used for the top coat can comprise, but are not limited to synthetic polymers such as poly(ethers), poly(ethylene glycol), poly(ethylene oxide), poly(propylene glycol), poly(lactams), poly(vinylpyrrolidone), poly(acrylates), poly(urethanes), poly(anhydrides), poly(amino acids), poly(carboxylic acids), poly(amides), poly(vinyl alcohol), and poly(phosphazenes).

Molecular weights of the hydrophilic polymers can range from, for example, about 500 amu to about 100,000 amu or from about 1,000 amu to about 40,000 amu.

Reactive groups, such as, but not limited to acrylates and/or methacrylates, can be added to the polymer via any convenient reactive moiety, such as hydroxyls, amines, or carboxylic acids, with a derivatization compound. In some embodiments, the derivatization compound can be a hetero-bifunctional compound. One moiety can react with the hydroxyl, amine, and/or carboxylic acid groups of the copolymer. The other moiety can be an acrylate or methacrylate group.

In some embodiments, the derivatization compound can comprise acryloyl chloride, methacryloyl chloride, 2-isocyanatoethyl acrylate, 2-isocyanatoethyl methacrylate, acrylic acid N-hydroxysuccinimide ester, methacrylic acid N-hydroxysuccinimide ester, hetero-bifunctional poly(ethylene glycol) with acrylate and isocyanate groups, combinations thereof, and derivatives thereof.

In embodiments, to prepare the derivatized polymer, the polymer, derivatization compound, and the optional catalyst are dissolved in a solvent. In general, any solvent that dissolves the top coat polymer, derivatization agent, and the optional catalyst can be used. Solvents can comprise aromatic and chlorinated solvents, including benzene, toluene, xylene, dichloromethane, chloroform, and combinations thereof.

In embodiments, when reacting a derivatization agent with a reactive moiety of the top coat polymer, the target derivatization corresponds to less than two groups per molecule. Additionally, in some embodiments, the derivatization can include addition of a Lewis base as a catalyst. In some embodiments, the Lewis base can be triethylamine and pyridine, in a concentration of about 1% to about 10% of the moles of the derivatization compound added.

In some embodiments, the derivatization reaction proceeds at, for example, room temperature.

After the derivatization is complete, an activated polymer can be recovered by precipitation in a non-solvent and dried under vacuum.

### Base Coat Application

In embodiments, the base coat can be applied to a medical device surface, such as a thermoplastic material. In embodiments, the device such as a catheter is first cleaned by a solvent wipe, for example to remove any gross contamination from its surface. In some embodiments, the catheter is wiped with a solvent. In some embodiments, any solvent can be used if it does not dissolve or degrade the thermoplastic material of the catheter. Solvents can include glycol ethers, methyl ethyl ketone, chlorinated solvents, tetrahydrofuran, hexane, ethyl acetate and acetone.

Following solvent cleaning, in some embodiments, the device such as a catheter shaft can be plasma treated to further clean its surface. In some embodiments, the catheter is not plasma treated. Plasmas derived from various gases can be used. In some embodiments, the plasma gases can be argon and oxygen. In some embodiments, both argon and oxygen plasmas can be used.

In embodiments, the base coat solution can comprise the solvent, base coat copolymer, an optional initiator, and an optional surfactant. Generally, any solvent or mixtures of solvents may be utilized, provided that the components can be dissolved into the solvent or solvent mixtures. Solvents can include water, alcohols, glycol ethers, aromatics, polar aprotic solvents, and combinations thereof. In some embodiments, the solvent can include methanol, ethanol, isopropyl alcohol, 2-ethoxy ethanol, propylene glycol monomethyl ether acetate, benzene, toluene, xylene, dimethyl formamide, dimethyl sulfoxide, and combinations thereof.

The base coat copolymer can be dissolved into the solvent at a concentration ranging from, for example, about 0.2% w/w to about 35% w/w, about 0.2% w/w to about 40% w/w, about 0.2% w/w to about 50% w/w, about 0.5% w/w to about 35% w/w, about 0.5% w/w to about 40% w/w, about 0.5% w/w to about 50% w/w, about 1% w/w to about 35% w/w, about 1% w/w to about 40% w/w, or about 1% w/w to about 50% w/w, depending on the desired viscosity of the basecoat solution. In some embodiments, the base coat copolymer concentration is about 15% w/w.

In some embodiments, if included, initiators can comprise Norrish Type I initiators, Norrish Type II initiators, and combinations thereof. Norrish Type I or free-radical photo-initiators can comprise benzoin derivatives, methylolbenzoin and 4-benzoyl-1,3-dioxolane derivatives, benzilketals, α,α-dialkoxyacetophenones, α-hydroxy alkylphenones, α-aminoalkylphenones, acylphosphine oxides, bisacylphosphine oxides, acylphosphine sulphides, halogenated acetophenone derivatives, or a combination thereof. In some embodiments, Norrish Type I photoinitiators can include Irgacure 2959 (2-hydroxy-4′-(2-hydroxyethoxy)-2-methyl propiophenone), Irgacure 651 (benzildimethyl ketal or 2,2-dimethoxy-1,2-diphenylethanone, Ciba-Geigy), Irgacure 184 (1-hydroxy-cyclohexyl-phenyl ketone as the active component, Ciba-Geigy), Darocur 1173 (2-hydroxy-2-methyl-1-phenylpropan-1-one as the active component, Ciba-Geigy), Irgacure 907 (2-methyl-1-[4-(methylthio)phenyl]-2-morpholino propan-1-one, Ciba-Geigy), Irgacure 369 (2-benzyl-2-dimethylamino-1-(4-morpholinophenyl)-butan-1-one as the active component, Ciba-Geigy), Esacure KIP 150 (poly {2-hydroxy-2-methyl-1-[4-(1-methylvinyl)phenyl]propan-1-one}, Fratelli Lamberti), Esacure KIP 100 F (blend of poly {2-hydroxy-2-methyl-1-[4-(1-methylvinyl)phenyl]propan-1-one} and 2-hydroxy-2-methyl-1-phenyl-propan-1-one, Fratelli Lamberti), Esacure KTO 46 (blend of poly {2-hydroxy-2-methyl-1-[4-(1-methylvinyl)phenyl]propan-1-one}, 2,4,6-trimethylbenzoyldiphenyl-phosphine oxide and methylbenzophenone derivatives, Fratelli Lamberti), acylphosphine oxides such as Lucirin TPO (2,4,6-trimethylbenzoyl diphenyl phosphine oxide, BASF), Irgacure 819 (bis(2,4,6-trimethylbenzoyl)-phenyl-phosphine-oxide, Ciba-Geigy), Irgacure 1700 (25:75% blend of bis(2,6-dimethoxybenzoyl)2,4,4-trimethyl-pentyl phosphine oxide and 2-hydroxy-2-methyl-1-phenyl-propan-1-one, Ciba-Geigy), or a combination thereof.

In some embodiments, mixtures of type I photo-initiators can be used.

In embodiments, Norrish Type II photo-initiators can also be used in the base coat formulation. These initiators can comprise aromatic ketones such as benzophenone, xanthone, derivatives of benzophenone (e.g. chlorobenzophenone), blends of benzophenone and benzophenone derivatives (e.g. Photocure 81, a 50/50 blend of 4-methyl-benzophenone and benzophenone), Michler's Ketone, Ethyl Michler's Ketone, thioxanthone and other xanthone derivatives like Quantacure ITX (isopropyl thioxanthone), benzil, anthraquinones (e.g. 2-ethyl anthraquinone), coumarin, or chemical derivatives or combinations of these photoinitiators.

In some embodiments, the base coat formulation can comprise combinations of Norrish Type I and Norrish Type II initiators.

In embodiments, the initiator concentration in the solvent can range from about 0.1% to about 6% w/w. In some embodiments, initiator concentration in the solvent can be about 0.6% w/w.

The base coat solution may also optionally comprise a surfactant. In some embodiments, any surfactant may be used. Surfactants can include sodium lauryl sulfate, Tween 20, Span 80, Triton X-100, Pluronic F68, Pluronic L-81, combinations thereof, and derivatives thereof. The optional surfactant can be dissolved into the selected solvent at a concentration ranging from about 0.1% w/w to about 15% w/w. In some embodiments, the surfactant concentration is about 0.8% w/w.

In some embodiments, to apply the base coat to a catheter, the length of the catheter desired to be coated is inserted into the base coat solution. In embodiments, the dip time, or amount of time the catheter spends in the base coat solution, ranges from about 0.2 to about 10 minutes, about 0.5 to about 10 minutes, about 2 to about 8 minutes, about 3 to about 6 minutes, or about 0.5 to about 8 minutes. In some embodiments, the dip time can be about 5 minutes.

In other embodiments, the base coat can be applied by, for example, spraying, brushing, spin coating, or the like, or a combination thereof including or not including dip coating.

In some embodiments, only portions or regions of the catheter are coated. Therein portions of the catheter can be masked so that base coat is not applied to the masked regions. For example, in embodiments, the coated portion of the device can comprise 0.1% of the surface area of the device, 0.2% of the surface area of the device, 0.3% of the surface area of the device, 0.4% of the surface area of the device, 0.5% of the surface area of the device, 0.6% of the surface area of the device, 0.7% of the surface area of the device, 0.8% of the surface area of the device, 0.9% of the surface area of the device, 1% of the surface area of the device, 1.5% of the surface area of the device, 2% of the surface area of the device, 2.5% of the surface area of the device, 3% of the surface area of the device, 5% of the surface area of the device, 10% of the surface area of the device, 15% of the surface area of the device, or more.

In embodiments, the coated portion of the device can comprise not more than 0.1% of the surface area of the device, not more than 0.2% of the surface area of the device, not more than 0.3% of the surface area of the device, not more than 0.4% of the surface area of the device, not more than 0.5% of the surface area of the device, not more than 0.6% of the surface area of the device, not more than 0.7% of the surface area of the device, not more than 0.8% of the surface area of the device, not more than 0.9% of the surface area of the device, not more than 1% of the surface area of the device, not more than 1.5% of the surface area of the device, not more than 2% of the surface area of the device, not more than 2.5% of the surface area of the device, not more than 3% of the surface area of the device, not more than 5% of the surface area of the device, not more than 10% of the surface area of the device, not more than 15% of the surface area of the device, or more.

Similarly, in embodiments, the coated portion of the device can comprise between about 0.1% to about 5% of the surface area of the device, about 0.2% to about 4.5% of the surface area of the device, about 0.3% to about 4% of the surface area of the device, about 0.4% to about 3.5% of the surface area of the device, about 0.5% to about 3% of the surface area of the device, about 0.6% to about 2.5% of the surface area of the device, about 0.7% to about 2% of the surface area of the device, about 0.8% to about 1.5% of the surface area of the device, about 0.9% to about 1% of the surface area of the device, or the like.

In further embodiments, the coated portion of the device can comprise between about 0.1% to about 20% of the surface area of the device, about 0.5% to about 15% of the surface area of the device, about 1% to about 10% of the surface area of the device, about 2% to about 9% of the surface area of the device, about 3% to about 8% of the surface area of the device, about 4% to about 6% of the surface area of the device, or the like.

In embodiments, the coated portion of the device can comprise not less than 0.1% of the surface area of the device, not less than 0.2% of the surface area of the device, not less than 0.3% of the surface area of the device, not less than 0.4% of the surface area of the device, not less than 0.5% of the surface area of the device, not less than 0.6% of the surface area of the device, not less than 0.7% of the surface area of the device, not less than 0.8% of the surface area of the device, not less than 0.9% of the surface area of the device, not less than 1% of the surface area of the device, not less than 1.5% of the surface area of the device, not less than 2% of the surface area of the device, not less than 2.5% of the surface area of the device, not less than 3% of the surface area of the device, not less than 5% of the surface area of the device, not less than 10% of the surface area of the device, not less than 15% of the surface area of the device, or less.

In embodiments, the coated portion comprises discrete portions separated by non-coated portions. For example, in embodiments, the coated portion comprises 1 discrete portion, 2 discrete portions, 3 discrete portions, 4 discrete portions, 5 discrete portions, 6 discrete portions, 7 discrete portions, 8 discrete portions, 9 discrete portions, 10 discrete portions, or more.

In embodiments, the coated portion comprises at least 1 discrete portion, at least 2 discrete portions, at least 3 discrete portions, at least 4 discrete portions, at least 5 discrete portions, at least 6 discrete portions, at least 7 discrete portions, at least 8 discrete portions, at least 9 discrete portions, at least 10 discrete portions, or more.

In embodiments, the coated portion comprises not more than 1 discrete portion, not more than 2 discrete portions, not more than 3 discrete portions, not more than 4 discrete portions, not more than 5 discrete portions, not more than 6 discrete portions, not more than 7 discrete portions, not more than 8 discrete portions, not more than 9 discrete portions, not more than 10 discrete portions, or the like.

In embodiments, the coated portion can comprise an “angled”, “bent”, or curved section of the device. For example, in embodiments, the angle can comprise an angle of at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 85 degrees, at least 90 degrees, at least 95 degrees, at least 100 degrees, at least 105 degrees, at least 110 degrees, at least 115 degrees, at least 120 degrees, at least 125 degrees, at least 130 degrees, at least 135 degrees, at least 140 degrees, at least 145 degrees, at least 150 degrees, at least 155 degrees, at least 160 degrees, at least 165 degrees, at least 170 degrees, at least 175 degrees, at least 180 degrees, at least 185 degrees, at least 190 degrees, at least 195 degrees, at least 200 degrees, at least 205 degrees, at least 210 degrees, at least 220 degrees, at least 225 degrees, at least 230 degrees, at least 235 degrees, at least 240 degrees, at least 245 degrees, at least 250 degrees, at least 255 degrees, at least 260 degrees, at least 265 degrees, at least 270 degrees, at least 275 degrees, at least 280 degrees, at least 285 degrees, at least 290 degrees, at least 295 degrees, at least 300 degrees, at least 305 degrees, at least 310 degrees, at least 315 degrees, at least 320 degrees, at least 325 degrees, at least 330 degrees, at least 335 degrees, at least 340 degrees, at least 345 degrees, at least 350 degrees, at least 355 degrees, or the like.

In further embodiments, the coated portion can comprise an angle of at most 10 degrees, at most 15 degrees, at most 20 degrees, at most 25 degrees, at most 30 degrees, at most 35 degrees, at most 40 degrees, at most 45 degrees, at most 50 degrees, at most 55 degrees, at most 60 degrees, at most 65 degrees, at most 70 degrees, at most 75 degrees, at most 80 degrees, at most 85 degrees, at most 90 degrees, at most 95 degrees, at most 100 degrees, at most 105 degrees, at most 110 degrees, at most 115 degrees, at most 120 degrees, at most 125 degrees, at most 130 degrees, at most 135 degrees, at most 140 degrees, at most 145 degrees, at most 150 degrees, at most 155 degrees, at most 160 degrees, at most 165 degrees, at most 170 degrees, at most 175 degrees, at most 180 degrees, at most 185 degrees, at most 190 degrees, at most 195 degrees, at most 200 degrees, at most 205 degrees, at most 210 degrees, at most 220 degrees, at most 225 degrees, at most 230 degrees, at most 235 degrees, at most 240 degrees, at most 245 degrees, at most 250 degrees, at most 255 degrees, at most 260 degrees, at most 265 degrees, at most 270 degrees, at most 275 degrees, at most 280 degrees, at most 285 degrees, at most 290 degrees, at most 295 degrees, at most 300 degrees, at most 305 degrees, at most 310 degrees, at most 315 degrees, at most 320 degrees, at most 325 degrees, at most 330 degrees, at most 335 degrees, at most 340 degrees, at most 345 degrees, at most 350 degrees, at most 355 degrees, or the like.

In further embodiments, the coated portion can comprise an angle of 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, 185 degrees, 190 degrees, 195 degrees, 200 degrees, 205 degrees, 210 degrees, 220 degrees, 225 degrees, 230 degrees, 235 degrees, 240 degrees, 245 degrees, 250 degrees, 255 degrees, 260 degrees, 265 degrees, 270 degrees, 275 degrees, 280 degrees, 285 degrees, 290 degrees, 295 degrees, 300 degrees, 305 degrees, 310 degrees, 315 degrees, 320 degrees, 325 degrees, 330 degrees, 335 degrees, 340 degrees, 345 degrees, 350 degrees, 355 degrees, or the like.

In further embodiments, the coated portion can comprise an angle of between about 10 and 350 degrees, between about 20 and 340 degrees, between about 30 and 330 degrees, between about 40 and 320 degrees, between about 50 and 310 degrees, between about 60 and 300 degrees, between about 70 and 290 degrees, between about 80 and 280 degrees, between about 90 and 270 degrees, between about 100 and 260 degrees, between about 110 and 250 degrees, between about 120 and 240 degrees, between about 130 and 230 degrees, between about 140 and 220 degrees, between about 150 and 210 degrees, between about 160 and 200 degrees, between about 170 and 190 degrees, or the like.

Similarly, in further embodiments, the coated portion can comprise an angle of between about 10 and 40 degrees, between about 20 and 50 degrees, between about 30 and 60 degrees, between about 40 and 70 degrees, between about 50 and 80 degrees, between about 60 and 90 degrees, between about 70 and 100 degrees, between about 80 and 110 degrees, between about 90 and 120 degrees, between about 100 and 130 degrees, between about 110 and 140 degrees, between about 120 and 150 degrees, between about 130 and 160 degrees, between about 140 and 170 degrees, between about 150 and 180 degrees, between about 160 and 190 degrees, between about 170 and 200 degrees, between about 180 and 210 degrees, between about 190 and 220 degrees, between about 200 and 230 degrees, between about 210 and 240 degrees, between about 220 and 250 degrees, between about 230 and 260 degrees, between about 240 and 270 degrees, between about 250 and 280 degrees, between about 260 and 290 degrees, between about 270 and 300 degrees, between about 280 and 310 degrees, between about 290 and 320 degrees, between about 300 and 330 degrees, between about 310 and 340 degrees, between about 320 and 350 degrees, or the like.

In embodiments, the coated portion can comprise a “bent” or curved section of the device flanked by two straight sections. For example, in embodiments, the straight sections may be joined by a bent or curved section wherein the straight sections, relative to each other, comprise an angle of at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 85 degrees, at least 90 degrees, at least 95 degrees, at least 100 degrees, at least 105 degrees, at least 110 degrees, at least 115 degrees, at least 120 degrees, at least 125 degrees, at least 130 degrees, at least 135 degrees, at least 140 degrees, at least 145 degrees, at least 150 degrees, at least 155 degrees, at least 160 degrees, at least 165 degrees, at least 170 degrees, at least 175 degrees, at least 180 degrees, at least 185 degrees, at least 190 degrees, at least 195 degrees, at least 200 degrees, at least 205 degrees, at least 210 degrees, at least 220 degrees, at least 225 degrees, at least 230 degrees, at least 235 degrees, at least 240 degrees, at least 245 degrees, at least 250 degrees, at least 255 degrees, at least 260 degrees, at least 265 degrees, at least 270 degrees, at least 275 degrees, at least 280 degrees, at least 285 degrees, at least 290 degrees, at least 295 degrees, at least 300 degrees, at least 305 degrees, at least 310 degrees, at least 315 degrees, at least 320 degrees, at least 325 degrees, at least 330 degrees, at least 335 degrees, at least 340 degrees, at least 345 degrees, at least 350 degrees, at least 355 degrees, or the like.

In further embodiments, the straight sections, relative to each other, can comprise an angle of at most 10 degrees, at most 15 degrees, at most 20 degrees, at most 25 degrees, at most 30 degrees, at most 35 degrees, at most 40 degrees, at most 45 degrees, at most 50 degrees, at most 55 degrees, at most 60 degrees, at most 65 degrees, at most 70 degrees, at most 75 degrees, at most 80 degrees, at most 85 degrees, at most 90 degrees, at most 95 degrees, at most 100 degrees, at most 105 degrees, at most 110 degrees, at most 115 degrees, at most 120 degrees, at most 125 degrees, at most 130 degrees, at most 135 degrees, at most 140 degrees, at most 145 degrees, at most 150 degrees, at most 155 degrees, at most 160 degrees, at most 165 degrees, at most 170 degrees, at most 175 degrees, at most 180 degrees, at most 185 degrees, at most 190 degrees, at most 195 degrees, at most 200 degrees, at most 205 degrees, at most 210 degrees, at most 220 degrees, at most 225 degrees, at most 230 degrees, at most 235 degrees, at most 240 degrees, at most 245 degrees, at most 250 degrees, at most 255 degrees, at most 260 degrees, at most 265 degrees, at most 270 degrees, at most 275 degrees, at most 280 degrees, at most 285 degrees, at most 290 degrees, at most 295 degrees, at most 300 degrees, at most 305 degrees, at most 310 degrees, at most 315 degrees, at most 320 degrees, at most 325 degrees, at most 330 degrees, at most 335 degrees, at most 340 degrees, at most 345 degrees, at most 350 degrees, at most 355 degrees, or the like.

In further embodiments, the straight sections, relative to each other, can comprise an angle of 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, 185 degrees, 190 degrees, 195 degrees, 200 degrees, 205 degrees, 210 degrees, 220 degrees, 225 degrees, 230 degrees, 235 degrees, 240 degrees, 245 degrees, 250 degrees, 255 degrees, 260 degrees, 265 degrees, 270 degrees, 275 degrees, 280 degrees, 285 degrees, 290 degrees, 295 degrees, 300 degrees, 305 degrees, 310 degrees, 315 degrees, 320 degrees, 325 degrees, 330 degrees, 335 degrees, 340 degrees, 345 degrees, 350 degrees, 355 degrees, or the like.

In further embodiments, the straight sections, relative to each other, can comprise an angle of between about 10 and 350 degrees, between about 20 and 340 degrees, between about 30 and 330 degrees, between about 40 and 320 degrees, between about 50 and 310 degrees, between about 60 and 300 degrees, between about 70 and 290 degrees, between about 80 and 280 degrees, between about 90 and 270 degrees, between about 100 and 260 degrees, between about 110 and 250 degrees, between about 120 and 240 degrees, between about 130 and 230 degrees, between about 140 and 220 degrees, between about 150 and 210 degrees, between about 160 and 200 degrees, between about 170 and 190 degrees, or the like.

Similarly, in further embodiments, the straight sections, relative to each other, can comprise an angle of between about 10 and 40 degrees, between about 20 and 50 degrees, between about 30 and 60 degrees, between about 40 and 70 degrees, between about 50 and 80 degrees, between about 60 and 90 degrees, between about 70 and 100 degrees, between about 80 and 110 degrees, between about 90 and 120 degrees, between about 100 and 130 degrees, between about 110 and 140 degrees, between about 120 and 150 degrees, between about 130 and 160 degrees, between about 140 and 170 degrees, between about 150 and 180 degrees, between about 160 and 190 degrees, between about 170 and 200 degrees, between about 180 and 210 degrees, between about 190 and 220 degrees, between about 200 and 230 degrees, between about 210 and 240 degrees, between about 220 and 250 degrees, between about 230 and 260 degrees, between about 240 and 270 degrees, between about 250 and 280 degrees, between about 260 and 290 degrees, between about 270 and 300 degrees, between about 280 and 310 degrees, between about 290 and 320 degrees, between about 300 and 330 degrees, between about 310 and 340 degrees, between about 320 and 350 degrees, or the like.

In embodiments, after dip coating or otherwise applying the base coat, the catheter is exposed to ultraviolet radiation with a wavelength ranging from, for example, about 10 nm to about 400 nm, about 100 nm to about 400 nm, about 200 nm to about 400 nm, about 200 nm to about 300 nm, or about 300 nm to about 400 nm. Combinations of wavelengths in this range can also provide a suitable base coat. In one embodiment, ultraviolet radiation can be applied by a first wavelength between about 200 nm to about 300 nm and a second wavelength between about 300 nm to about 400 nm. In one embodiment, wavelengths can include 254 and 365 nm.

In embodiments, the cure time, or amount of time the catheter is exposed to ultraviolet radiation, ranges from about 0.5 to about 10 minutes, about 1 to about 10 minutes, about 1 to about 8 minutes, about 0.5 to about 6 minutes, about 1 to about 6 minutes, about 1 to about 3 minutes, or about 0.5 to about 30 minutes. In one embodiment, the cure time is about 2 minutes.

In some embodiments, the base coat application process is complete after the completion of the cure time.

### Top Coat Application

In embodiments, the top coat can be applied to a completed base coat. The top coat solution can comprise the solvent, a top coat polymer, an optional initiator, and an optional surfactant. In general, any solvent or mixtures of solvents may be utilized, provided that the components can be dissolved into the solvent or solvent mixtures. Suitable solvents can comprise water, alcohols, glycol ethers, aromatics, polar aprotic solvents, and combinations thereof. In some embodiments, the solvent can comprise methanol, ethanol, isopropyl alcohol, 2-ethoxy ethanol, propylene glycol monomethyl ether acetate, benzene, toluene, xylene, dimethyl formamide, dimethyl sulfoxide, and combinations thereof.

In embodiments, the top coat polymer can be dissolved into the selected solvent at a concentration ranging from about 5% w/w to about 75% w/w, about 5% w/w to about 80% w/w, about 5% w/w to about 90% w/w, about 10% w/w to about 80% w/w, about 10% w/w to about 75% w/w, about 5% w/w to about 50% w/w, about 5% w/w to about 40% w/w, about 5% w/w to about 40% w/w, about 20% w/w to about 40% w/w, about 20% w/w to about 30% w/w, depending on the desired viscosity of the top coat solution. In one embodiment, the top coat polymer concentration is about 25% w/w.

In embodiments, the optional initiator can comprise Norrish Type I initiators, Norrish Type II initiators, and combinations thereof. Norrish Type I or free-radical photo-initiators can include benzoin derivatives, methylolbenzoin and 4-benzoyl-1,3-dioxolane derivatives, benzilketals, α,α-dialkoxyacetophenones, α-hydroxy alkylphenones, α-aminoalkylphenones, acylphosphine oxides, bisacylphosphine oxides, acylphosphine sulphides, halogenated acetophenone derivatives, and the like. Norrish Type I photoinitiators can include Irgacure 2959 (2-hydroxy-4′-(2-hydroxyethoxy)-2-methyl propiophenone), Irgacure 651 (benzildimethyl ketal or 2,2-dimethoxy-1,2-diphenylethanone, Ciba-Geigy), Irgacure 184 (1-hydroxy-cyclohexyl-phenyl ketone as the active component, Ciba-Geigy), Darocur 1173 (2-hydroxy-2-methyl-1-phenylpropan-1-one as the active component, Ciba-Geigy), Irgacure 907 (2-methyl-1-[4-(methylthio)phenyl]-2-morpholino propan-1-one, Ciba-Geigy), Irgacure 369 (2-benzyl-2-dimethylamino-1-(4-morpholinophenyl)-butan-1-one as the active component, Ciba-Geigy), Esacure KIP 150 (poly {2-hydroxy-2-methyl-1-[4-(1-methylvinyl)phenyl]propan-1-one}, Fratelli Lamberti), Esacure KIP 100 F (blend of poly {2-hydroxy-2-methyl-1-[4-(1-methylvinyl)phenyl]propan-1-one} and 2-hydroxy-2-methyl-1-phenyl-propan-1-one, Fratelli Lamberti), Esacure KTO 46 (blend of poly {2-hydroxy-2-methyl-1-[4-(1-methylvinyl)phenyl]propan-1-one}, 2,4,6-trimethylbenzoyldiphenyl-phosphine oxide and methylbenzophenone derivatives, Fratelli Lamberti), acylphosphine oxides such as Lucirin TPO (2,4,6-trimethylbenzoyl diphenyl phosphine oxide, BASF), Irgacure 819 (bis(2,4,6-trimethylbenzoyl)-phenyl-phosphine-oxide, Ciba-Geigy), Irgacure 1700 (25:75% blend of bis(2,6-dimethoxybenzoyl)2,4,4-trimethyl-pentyl phosphine oxide and 2-hydroxy-2-methyl-1-phenyl-propan-1-one, Ciba-Geigy), and the like. Also, mixtures of type I photo-initiators can be used.

Norrish Type II photo-initiators that can be used comprise aromatic ketones such as benzophenone, xanthone, derivatives of benzophenone (e.g. chlorobenzophenone), blends of benzophenone and benzophenone derivatives (e.g. Photocure 81, a 50/50 blend of 4-methyl-benzophenone and benzophenone), Michler's Ketone, Ethyl Michler's Ketone, thioxanthone and other xanthone derivatives like Quantacure ITX (isopropyl thioxanthone), benzil, anthraquinones (e.g. 2-ethyl anthraquinone), coumarin, or chemical derivatives or combinations thereof.

In some embodiments, the top coat formulation can comprise combinations of Norrish Type I and Norrish Type II initiators.

In embodiments, the initiator concentration in the solvent can range from about 0.1% to about 6% w/w. In some embodiments, initiator concentration in the solvent can be about 0.5% w/w.

In embodiments, the top coat solution may also comprise a surfactant. In general, any surfactant may be used. In some embodiments, surfactants can include sodium lauryl sulfate, Tween 20, Span 80, Triton X-100, Pluronic F68, Pluronic L-81, combinations thereof, and derivatives thereof. The optional surfactant can be dissolved into the selected solvent at a concentration ranging from about 0.1% w/w to about 5% w/w. In some embodiments, the surfactant concentration is about 0.6% w/w.

In some embodiments, to apply the top coat to a base coated catheter, the length of the catheter desired to be coated is inserted into the base coat solution. In embodiments, the “dip” time, or amount of time the catheter spends in the base coat solution, ranges from about 0.2 to about 20 minutes, about 0.5 to about 20 minutes, about 2 to about 15 minutes, about 3 to about 15 minutes, or about 8 to about 12 minutes. In some embodiments, the dip time can be about 10 minutes.

In other embodiments, the top coat can be applied by spraying, brushing, spin coating, or the like, or a combination thereof including or not including dip coating.

In some embodiments, only portions of the catheter are coated with the top coat. Therein portions of the catheter can be masked so that top coat is not applied to the masked regions.

In embodiments, after dip coating or otherwise applying the top coat, the catheter is exposed to ultraviolet radiation with a wavelength ranging from about 10 nm to about 400 nm, about 100 nm to about 400 nm, about 200 nm to about 400 nm, about 200 nm to about 300 nm, or about 300 nm to about 400 nm. Combinations of wavelengths in this range can also provide a suitable base coat. In one embodiment, ultraviolet radiation can be applied by a first wavelength between about 200 nm to about 300 nm and a second wavelength between about 300 nm to about 400 nm. In one embodiment, wavelengths can include 254 and 365 nm.

The top coat cure time, or amount of time the catheter is exposed to ultraviolet radiation, ranges from about 0.5 to about 4 minutes, about 1 to about 4 minutes, about 1 to about 3 minutes, about 0.5 to about 3 minutes, about 1 to about 5 minutes, about 0.5 to about 3 minutes, or about 0.5 to about 50 minutes. In one embodiment, the cure time is about 2 minutes.

The herein described coatings can provide a reduction in maximum dynamic friction force [gf] when compared to an uncoated device. In some embodiments, the coatings can reduce the maximum dynamic friction force by about, for example, 10%, 20%, 30%, 40%, 50%, 60%, or more. In other embodiments, the coatings can reduce the maximum dynamic friction force by about 75%.

The herein described coatings can provide a reduction in average dynamic friction force at 60 mm displacement for 100 cycles [gf] when compared to an uncoated device. In some embodiments, the coatings can reduce the maximum dynamic friction force by about, for example, 10%, 20%, 30%, 40%, 50%, 60%, or more. In other embodiments, the coatings can reduce the maximum dynamic friction force by about 75%.

The herein described coatings can provide an increase in lubricity when compared to an uncoated device. In some embodiments, the coatings can increase the lubricity by about, for example, 10%, 15%, 20%, 25%, 30%, 35%, 40%, 45%, 50%, 55%, 60%, 65%, 70%, 75%, 80%, 85%, or the like.

In some embodiments, the coatings can increase the lubricity by about, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or the like.

The coatings described herein are applied to the inner support catheter. In some embodiments, the inner support catheter can be partially coated. In other embodiments, the inner support catheter can be completed coated. In some embodiments, a portion or section of the inner support catheter is coated. The coated section can be a bulbous section of the inner support catheter. In other embodiments, the coated section can be a non-bulbous section of the inner support catheter. In other embodiments, the specific section where friction is observed is coated. In some embodiments, the specific section can vary in length depending upon the amount of friction observed.

In some embodiments, the coating is not applied to a distal section of the catheter. For example, other devices have coatings applied across the entire distal section of the catheter which can help reduce friction. However, when the coating is applied to a distal section of the inner support catheter, the inner support catheter loses position when advancing a guidewire through the lumen, or a guide catheter advancing over the inner support catheter. Coatings applied to a distal end of an inner support catheter can help reduce friction, however stability is also reduced. As such, the coatings described herein applied to a specific section of the inner support catheter can provide a reduction in friction without reducing stability.

The coatings described herein can be applied to a specific section of the catheter. In some embodiments, the specific section can be the specific section where friction is observed. In other embodiments, the specific section can be at the proximal end of the catheter. In some embodiments, the specific section can be at a constricted, angled, or bulbous region/section of the catheter.

In some embodiments, a method for applying a coating to a specific section of a catheter is described wherein the coating comprises a base coat including a copolymer of a first tetrahydrofuryl acrylate monomer and a second monomer including a functional group amenable to further derivatization and plurality of reactive moieties, and a top coat including a hydrophilic polymer containing more than two reactive moieties per molecule is described. In other embodiments, a method for applying a coating to a bulbous section of an inner support catheter is described wherein the coating comprises a base coat including a copolymer of a first tetrahydrofuryl acrylate monomer and a second monomer including a functional group amenable to further derivatization and plurality of reactive moieties, and a top coat including a hydrophilic polymer containing more than two reactive moieties per molecule is described.

### Example 1

### Preparation of a Base Coat Polymer

To a 1 L round bottom flask are added 80.0 g of tetrahydrofurfuryl acrylate, 18.5 g of 4-hydroxybutyl acrylate and 250 mL of toluene. The solution is de-gassed by purging argon gas through for 30 min. Then, 1.0 gram AIBN initiator is added, and the mixture is purged with argon for another 10 min. The flask is immersed in an 80° C. oil bath and reflux condenser with argon inlet attached. The mixture is heated for 16 hours under argon. The reaction is cooled down and precipitated with 1.2 L of cold MTBE, precipitated product—viscous polymer is collected and dried at vacuum. Typical yield is 85-95%.

The dried polymer is dissolved in dry DMF (200 mL, about 0.5 g/mL) and treated with 0.84 mL of triethylamine and 3.0 mL of isocyanatoethyl acrylate. The mixture is heated to 45° C. for 5 hrs. The polymer is precipitated out with 1.2 L of cold MTBE, washed 2×200 ml of MTBE and dried at high vacuum.

### Example 2

### Preparation of a Liquid Base Coat Solution

In an appropriate container, 6.75 g of polymer from Example 1 is dissolved in 45.0 mL of propylene glycol monomethyl ether acetate. Then, 0.34 g of Pluronic L-81 surfactant, 135 mg of benzophenone, and 135 mg 1-hydroxycyclohexyl phenyl ketone are added. Complete dissolution is achieved with shaking for 30 minutes produces a clear, homogeneous solution.

### Example 3

Coating a Microcatheter with a Base Coat Solution

A 12 inch length (0.027″ outer diameter) section of microcatheter with an external surface comprised of Grilamid L25 is prepared for coating by first inserting a tightly fitting stainless steel mandrel into the hollow inner lumen, then wiping the outer surface with acetone. The microcatheter section is then plasma treated with argon plasma (365 sccm, 300 watts, 500 mtorr) followed by oxygen plasma (120 sccm, 150 watts, 400 mtorr). The liquid base coat formulation prepared in Example 2 is transferred into a glass tube (12″ length, 0.22″ inner diameter) with a stoppered bottom. The microcatheter section is then placed into the coating solution in the glass tube and allowed to dwell for 5 minutes. The microcatheter section is removed and immediately cured by UV radiation (254 nm λ, 1.3 J/cm2 UV dose) over 2 minutes to polymerize the base coat onto the Grilamid substrate.

### Example 4

### Preparation of a Top Coat Macromer

Fifty (50) grams of PEG (Mw 4,000) is dried by azeotropic distillation with toluene. A solution of PEG in 250 ml of toluene is treated with 30 mL of dichloromethane, followed by addition of 7.0 mL of triethylamine and 4.04 mL of acryloyl chloride. The reaction mixture is stirred for 5 hrs and then precipitated salts are filtered off and top coat macromer is isolated by precipitation from 1 L of cold MTBE. Solids are separated by filtration, washed with additional 200 mL of MTBE and dried at high vacuum overnight.

### Example 5

### Preparation of a Top Coat Solution

In a container, 9.0 g of polyethylene glycol di-acrylate (4,000 Mw) prepared in Example 4 is dissolved in 45.0 mL of methanol with shaking. Then, 0.23 g of Pluronic L-81 surfactant, 90 mg of benzophenone, and 90 mg of 1-hydroxycyclohexyl phenyl ketone are added. Complete dissolution with heating at 55° C. for 2 minutes results in a clear, homogenous solution.

### Example 6

Coating Microcatheter with a Top Coat Solution

The top coat solution prepared in Example 5 is transferred into a glass tube with a stoppered bottom and the microcatheter section with base coat from Example 3 is placed into the glass tube and allowed to dwell for 10 minutes in the top coat solution. The microcatheter section is then removed and immediately cured by UV radiation (254 nm λ, 1.3 J/cm2 UV dose) over 2 minutes to polymerize the top coat onto the base coat.

### Example 7

### Lubricity

Microcatheter samples prepared in Example 6 are tested to evaluate lubricity using an Instron 5943 material tester equipped with a 5 N static load cell. A mechanical clamping fixture is attached to the load cell to hold the top of the microcatheter sample as its length is pulled through a hydraulic clamping fixture (clamping force of 1 lb.) submerged in a heated (37° C.) water bath containing distilled water. The test method cycles each sample repeatedly 20 times at a pull rate of 254 mm/min for 100 mm, with one cycle measured as starting at 0 mm displacement with the hydraulic clamp closed on the sample. Then, the sample is pulled through the hydraulic clamp for 100 mm displacement, and finally the hydraulic clamp is opened, and the sample is returned to 0 mm displacement. The maximum dynamic friction force and the average dynamic friction force at the 60 mm displacement mark is measured and presented in the Table below. Included in the table are lubricity measurement results for an uncoated sample run for 20 cycles as a comparison.

| | Maximum | Avg. Dynamic |
|---|---|---|
| | Dynamic | Friction Force at |
| Sample | Friction Force | 60 mm Displacement |
| Number | [gf] | for 100 cycles [gf] |
| 1 | 189.0 | 157.7 |
| 2 | 115.0 | 106.0 |
| Uncoated | 411 | 305 |

The coating of example 6 compared to an uncoated sample illustrates an increase in lubricity.

### DISCLOSED EMBODIMENTS

Embodiment 1) A method of reducing the maximum dynamic friction force [gf] of a medical device comprising:
applying a base coat to the device;
applying a top coat to the base coat;
wherein said base coat and top coat form a lubricious surface on said medical device, thereby reducing the maximum dynamic friction force.

Embodiment 2) The method of embodiment 1, wherein said base coat and top coat are applied to at least 1% of the surface area of the device

Embodiment 3) The method of embodiment 2, wherein said base coat and top coat are applied to at least 5% of the surface area of the device.

Embodiment 4) The method of embodiment 3, wherein said base coat and top coat are applied to at least 10% of the surface area of the device.

Embodiment 5) The method of embodiment 2, wherein said base coat comprises a copolymer of a first tetrahydrofuryl acrylate monomer and a second monomer.

Embodiment 6) The method of embodiment 5, wherein said second monomer comprises hydroxyl, amine, or carboxylic acid groups.

Embodiment 7) The method of embodiment 6, wherein said second monomer comprises hydroxyethyl methacrylate, hydroxyethyl acrylate, hydroxypropyl acrylate, hydroxypropyl methacrylate, hydroxybutyl acrylate, hydroxybutyl methacrylate, N-(3-aminopropyl) methacrylamide, 2-aminoethyl methacrylate, 2-aminoethyl methacrylamide, acrylic acid, methacrylic acid, beta-carboxyethyl acrylate, combinations thereof, or derivatives thereof.

Embodiment 8) The method of embodiment 1, wherein said top coat comprises proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, dextran, poly(ethers), poly(ethylene glycol), poly(ethylene oxide), poly(propylene glycol), poly(lactams), poly(vinylpyrrolidone), poly(acrylates), poly(urethanes), poly(anhydrides), poly(amino acids), poly(carboxylic acids), poly(amides), poly(vinyl alcohol), or poly(phosphazenes).

Embodiment 9) The method of embodiment 1, wherein said base coat is applied to an angled, constricted, or bulbous portion of the medical device.

Embodiment 10) The method of embodiment 5, wherein said base coat is made by dissolving said copolymer and said monomer in a solvent selected from benzene, toluene, xylene, dimethylformamide, dimethyl sulfoxide, dioxane, 2-methyltetrahydrofuran, anisole, benzonitrile, chlorinated aromatic solvents, diisopropyl ether, diglyme, butanol, and combinations thereof.

Embodiment 11) A medical device comprising an angled, bent, or constricted region, said angled, bent, or constricted region comprising a lubricious coating.

Embodiment 12) The medical device of embodiment 11, wherein said angled, bent, or constricted region is flanked by straight regions.

Embodiment 13) The medical device of embodiment 12, wherein said straight regions, relative to each other, form an angle of at least 10 degrees.

Embodiment 14) The medical device of embodiment 13, wherein said device comprises a catheter.

Embodiment 15) The medical device of embodiment 14, wherein said straight regions comprises an angle of at least 15 degrees.

Embodiment 16) The medical device of embodiment 15, wherein said straight regions comprises an angle of at least 20 degrees.

Embodiment 17) The medical device of embodiment 11, wherein said lubricious coating comprises a base coat.

Embodiment 18) The medical device of embodiment 17, wherein said lubricious coating comprises a top coat.

Embodiment 19) The medical device of embodiment 18, wherein said lubricious coating reduces the maximum dynamic friction force [gf] of the device by 10% when compared to an uncoated device.

Embodiment 20) The medical device of embodiment 18, wherein said lubricious coating reduces the maximum dynamic friction force [gf] of the device by 20% when compared to an uncoated device.

Embodiment 21) The medical device of embodiment 17, wherein said base coat comprises a copolymer of a first tetrahydrofuryl acrylate monomer and a second monomer comprising hydroxyl, amine, or carboxylic acid groups.

Embodiment 22) The medical device of embodiment 18, wherein said top coat comprises proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, dextran, poly(ethers), poly(ethylene glycol), poly(ethylene oxide), poly(propylene glycol), poly(lactams), poly(vinylpyrrolidone), poly(acrylates), poly(urethanes), poly(anhydrides), poly(amino acids), poly(carboxylic acids), poly(amides), poly(vinyl alcohol), or poly(phosphazenes).

Although preferred embodiments have been described in this specification and the accompanying drawings, it will be appreciated that a number of variations and modifications may suggest themselves to those skilled in the pertinent arts. Thus, the scope of the present invention is not limited to the specific embodiments and examples described herein, but should be deemed to encompass alternative embodiments and equivalents.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1) A method of reducing the maximum dynamic friction force [gf] of a medical device comprising:
applying a base coat to an angled, constricted, or bulbous portion of the device;
applying a top coat to the base coat;
wherein said base coat and top coat form a lubricious surface on said medical device, thereby reducing the maximum dynamic friction force.

2) The method of claim 1, wherein said base coat and top coat are applied to at least 1% of the surface area of the device.

3) The method of claim 2, wherein said base coat and top coat are applied to at least 5% of the surface area of the device.

4) The method of claim 3, wherein said base coat and top coat are applied to at least 10% of the surface area of the device.

5) The method of claim 2, wherein said base coat comprises a copolymer of a first tetrahydrofuryl acrylate monomer and a second monomer.

6) The method of claim 5, wherein said second monomer comprises hydroxyl, amine, or carboxylic acid groups.

7) The method of claim 6, wherein said second monomer comprises hydroxyethyl methacrylate, hydroxyethyl acrylate, hydroxypropyl acrylate, hydroxypropyl methacrylate, hydroxybutyl acrylate, hydroxybutyl methacrylate, N-(3-aminopropyl) methacrylamide, 2-aminoethyl methacrylate, 2-aminoethyl methacrylamide, acrylic acid, methacrylic acid, beta-carboxyethyl acrylate, combinations thereof, or derivatives thereof.

8) The method of claim 1, wherein said top coat comprises proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, dextran, poly(ethers), poly(ethylene glycol), poly(ethylene oxide), polypropylene glycol), poly(lactams), poly(vinylpyrrolidone), poly(acrylates), poly(urethanes), poly(anhydrides), poly(amino acids), poly(carboxylic acids), poly(amides), poly(vinyl alcohol), or poly(phosphazenes).

9) (canceled)

10) The method of claim 5, wherein said base coat is made by dissolving said copolymer and said monomer in a solvent selected from benzene, toluene, xylene, dimethylformamide, dimethyl sulfoxide, dioxane, 2-methyltetrahydrofuran, anisole, benzonitrile, chlorinated aromatic solvents, diisopropyl ether, diglyme, butanol, and combinations thereof.

11) A medical device comprising an angled, bent, or constricted region, said angled, bent, or constricted region comprising a lubricious coating, said lubricious coating comprising:
a base coat; and
a top coat.

12) (canceled)

13) (canceled)

14) (canceled)

15) (canceled)

16) (canceled)

17) (canceled)

18) (canceled)

19) The medical device of claim 11, wherein said lubricious coating reduces the maximum dynamic friction force [gf] of the device by 10% when compared to an uncoated device.

20) The medical device of claim 11, wherein said lubricious coating reduces the maximum dynamic friction force [gf] of the device by 20% when compared to an uncoated device.

21) The medical device of claim 11, wherein said base coat comprises a copolymer of a first tetrahydrofuryl acrylate monomer and a second monomer comprising hydroxyl, amine, or carboxylic acid groups.

22) The medical device of claim 11, wherein said top coat comprises proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, dextran, poly(ethers), poly(ethylene glycol), poly(ethylene oxide), polypropylene glycol), poly(lactams), poly(vinylpyrrolidone), poly(acrylates), poly(urethanes), poly(anhydrides), poly(amino acids), poly(carboxylic acids), poly(amides), poly(vinyl alcohol), or poly(phosphazenes).

23) A method of catheterizing a patient in need thereof, said method comprising use of a catheter comprising an angled, bent, or constricted region, said angled, bent, or constricted region comprising a lubricious coating, said lubricious coating comprising:
a base coat; and
a top coat.

24) The method of claim 23, wherein said lubricious coating reduces the maximum dynamic friction force [gf] of the device by 10% when compared to an uncoated device.

25) The method of claim 24, wherein said lubricious coating reduces the maximum dynamic friction force [gf] of the device by 20% when compared to an uncoated device.

26) The method of claim 25, wherein said base coat comprises a copolymer of a first tetrahydrofuryl acrylate monomer and a second monomer comprising hydroxyl, amine, or carboxylic acid groups.

27) The method of claim 26, wherein said top coat comprises proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, dextran, poly(ethers), poly(ethylene glycol), poly(ethylene oxide), polypropylene glycol), poly(lactams), poly(vinylpyrrolidone), poly(acrylates), poly(urethanes), poly(anhydrides), poly(amino acids), poly(carboxylic acids), poly(amides), poly(vinyl alcohol), or poly(phosphazenes).
